(19) United States
(12) Patent Application Publication
(10) Pub. No.: US 2010/0106061 A1
(43) Pub. Date: Apr. 29, 2010

(54) METHOD AND ARRANGEMENT FOR THE THREE-DIMENSIONAL DETECTION OF THE SPATIAL SHAPE OF A FOOT

(75) Inventors: Christian Lott, Kaiserslautern (DE); Dirk Rutschmann, Stuttgart (DE)

Correspondence Address: LAW OFFICES OF STUART J. FRIEDMAN, 28930 RIDGE ROAD, MT. AIRY, MD, 21771, US

(73) Assignee: corpus.e AG, Stuttgart (DE)

(21) Appl. No.: 12/448,665

(22) PCT Filed: Dec. 19, 2007
(86) PCT No.: PCT/EP2007/011196

§ 371 Date: Nov. 27, 2009

(30) Foreign Application Priority Data
Jan. 1, 2007 (DE) 10 2007 001 402.5

(51) Int. Cl. A61B 5/107

(52) U.S. Cl. 600/592

(57) ABSTRACT

A method for the three-dimensional, digitized sensing of the spatial shape of at least one foot of a person. The method includes: determining a desired position for the foot which is predefined by the footwear to be selected or fabricated; moving the foot to the desired position by means of a device which can preferably be adjusted using a motor; optical sensing and digitizing the three-dimensional spatial shape of the foot in the desired position. Furthermore, an arrangement by means of which at least one foot of a person is moved to a desired position and the spatial shape of the foot is digitized in a mainly contactless manner includes a bottom plate, a first support and a second support. The first and second supports are movable, preferably using a motor, parallel or perpendicular to the bottom plate to move the foot to the desired position.

The invention relates to a method for the three-dimensional, digitized sensing of the spatial shape of at least one foot or of both feet of a person for a selection or individual manufacture of footwear. The invention further relates to an arrangement by means of which a foot or both feet of a person is/are brought into a desired position in which the spatial shape of the foot/feet is then digitized in a mainly contactless manner.

The sensing of the spatial shape, i.e. of the three-dimensional coordinates of the surface of feet for the selection of fitting products or for the dimensionally accurate manufacture of these products, for the acquisition of anatomically and medically interesting data and similar applications, is nowadays preferably carried out with optical 3D scanners which operate on the basis of laser triangulation, projection of structured light or near range photogrammetry. A particularly cost-effective arrangement of such a scanner is the so-called “Lightbeam® 3D Scanner” of corpus.e AG, Stuttgart (www.corpus-e.com). Here, the foot to be digitized is covered with a specially marked elastic, tight-fitting cover and photographed using a digital camera from several views overlapping each other. The photogrammetric recording, i.e. the allocation of corresponding marks from the individual images is carried out with the special encoding of these marks as well as an equally photogrammetrically marked bottom plate on which the customer to be digitized stands upright. This method is described in a series of granted patents and patent applications such as for instance in basic patent EP 0 760 622: Method and Arrangement for the Three-Dimensional, Digitized Sensing of the Spatial Shape of Bodies or Body Parts (inventor: Robert Massen).

A further foot scanner commercially offered by the Shoemaster company under the name “INFOOT®” (cf. www.shoemaster.co.uk) uses the laser light section method, wherein only one foot at a time, which is positioned on a glass plate, is scanned by means of an arrangement comprised of three movable camera/laser line projector arrangements.

In the prior art, an only slightly controlled actual state of the shape of the feet, which in principle have a very high dimensional instability, is measured in the three-dimensional digitalization: the customer stands upright on a generally flat bottom plate (for example a glass plate in laser triangulation scanners which also digitize the sole of the foot, or a photogrammetrically marked surface in the Lightbeam® Scanner).

Under the influence of the body weight and the posture, the digitized foot takes a spatial shape which considerably differs from the later foot shape within a well-fitting shoes: The sole of the foot is more or less flattened depending on the body balance posture, and the peripheral dimensions and length dimensions clearly differ from those of a foot in the walking movement.

In particular in women's shoes with a high heel, the foot shape in the normal use, due to the steep spatial position, clearly differs from the foot shape which is determined in a flat basic position in an optical scanner according to the prior art described. The data extracted from a 3D model of a foot digitized in this position are therefore possibly hardly suitable to choose an appropriate model or to manufacture an individual shoe. These difficulties, among other things, led to the fact that up to now, practically no women's shoes chosen or individually manufactured using 3D scan data have been offered in the so-called mass-customization business.

However, for the different uses of the foot digitalization in the field of prosthetic care, in the field of the so-called mass-customization and in special shoes such as sports shoes, protective shoes, etc., there is a great interest to bring the foot to be digitized into an anatomically desired position before measurement to then digitize the foot thus fixed in a defined desired state. Here, this desired position can be defined not only by a specific geometric spatial position, for example by a specific angle between the sole of the foot and the front toe plateau, but also by reaching specific physical features such as, e.g., a specific pressure distribution of the foot sole pressure from the heel to the ball.

In particular in prosthetic shoes, which apart from the actual footwear also constitute an auxiliary means for the correction of symptoms having no relation to the foot, such as, e.g. an oblique position of the pelvis, it is very important to derive this shoe from a foot model which is digitized in the right correction position.

Therefore, there is a considerable economic and technical interest in a method for the acquisition of the spatial shape of feet with 3D-scanners operating in a contactless manner, in which the foot model produced is adapted to the footwear to be selected or manufactured.

According to the invention, this is achieved by a method comprising the steps of claim 1. Furthermore, by an arrangement having the features of claim 11, it is achieved that the foot to be digitized is moved to a desired position adapted to the footwear.

Due to the digitalization of the foot in a desired spatial position it is achieved that the 3D model of the foot produced by the 3D digitalization provides the data required for the selection of a best-fit shoe from an existing collection or for the manufacture of an individual custom-made shoe much more accurately than is possible with the 3D-model of a foot, for example of a female foot which is in a flat position and therefore deforms in different ways. Since derived usual length and peripheral dimensions can also change up to several shoe sizes due to the foot deformation accompanying the changed spatial position, it is directly obvious that a digitalization of a foot in such an optimum spatial position leads to shoes having a considerably better fit.

This argumentation is also decisive in the digitalization for producing orthopaedic shoes, so that in this field the method according to the invention also provides considerably better fitting 3D models and length and peripheral dimensions derived therefrom than are produced in foot scanners according to the prior art.

Advantageously, the foot or the feet is/are successively moved to different desired positions, and the three-dimensional spatial shape is determined in each of these positions. It is therefore possible to take deformations of the foot, for example during walking, into consideration. Preferably, the supports moving the foot to the desired position are adjusted using a motor.

In the preferred embodiment, the arrangement by means of which the foot is moved to the desired position, includes pressure sensors, and the desired position is at least also determined by a predefined pressure distribution. It is therefore possible, for example, to evaluate positions desired by the customer from an anatomical point of view, and the customer can be advised accordingly.

The inventive idea is to be illustrated by way of example but not in a restricting manner with reference to the digitalization of a female foot, a two-part device adjustable by a motor being used to raise the arch of the foot and the heel of the foot, and the resting pressure on the three points, i.e. on the front part of the foot, the arch of the foot and the heel of the foot, being determined by pressure sensors mounted in the device. Here, reference is made to the following illustrations:

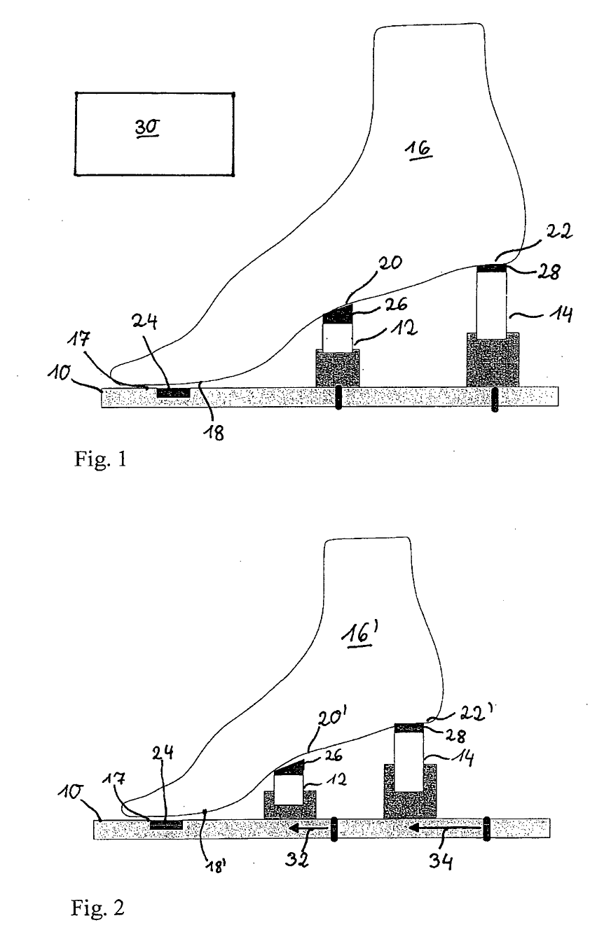

FIG. 1 shows an arrangement according to the invention when digitizing a first foot, and

FIG. 2 shows the arrangement according to the invention of FIG. 1 when digitizing a second foot.

The arrangement according to the invention in FIG. 1 includes a bottom plate 10, a first support 12 and a second support 14. The supports and the bottom plate are preferably made of a transparent material so that they have a disturbing influence that is as small as possible when optically sensing the spatial shape, and so that they do not limit the digitizing region or limit the latter only slightly. Depending on the digitizing method used, the bottom plate comprises photogrammectric marks. The inventive idea is independent of the mainly contactless and optical digitizing method used, be it on the basis of laser triangulation, pattern projection, photogrammetry, silhouette sensing or other methods known to a person skilled in the art in the field of 3D digitizing.

The front part of the foot 16 to be digitized rests on a support surface 17, the arch 20 thereof rests on the first support 12 and the heel 22 thereof rests on the second support 14. The supports are preferably to be changed pneumatically or hydraulically or in a motor controlled manner. In the embodiment, the supports 12 and 14 are marked (not shown) such that they can be identified by the digitizing system. This can be performed for example by an optical encoding with a bar code, with an RFID chip or a similar marking method, or by a shape recognition of the supports themselves, which are also digitized.

It is also conceivable to move the foot to the desired position using mechanical positioning devices which are not made up of discrete vertical axes, as shown in FIG. 1, but position the sole of the foot over an area in the space. Such positioning devices can for example be made up of inflatable flexible hollow bodies. Simplified configurations of the devices, for example in the form of a group of different foot supports corresponding to the different spatial positions, are also conceivable.

In the embodiment, a pressure sensor 24 is provided in the support surface 16, a pressure sensor 26 is provided in the first support 12, and a pressure sensor 28 is provided in the second support 14. The supports 12 and 14 can be moved vertically. In FIG. 1, they are adjusted such that the female foot is moved to a desired position which corresponds to the foot posture corresponding to the desired fashionable shoe style, and to the anatomically desired pressure ratios present between the resting pressure of the front part 18 of the foot (for example at the point of the ball), the arch 20 of the foot and the heel 22. Advantageously, sensors for static and/or dynamic pressure values and/or temperature values and/or electrodynamic values at different points of the foot sole can also be provided. The supports 12 and 14 are moved vertically by a motor, however, a mechanical adjustment is also conceivable to reach the desired position.

Each support 12 and 14 preferably includes two separately movable devices arranged next to each other, by means of which, due to different heights of the left-hand and right-hand devices, a tilting of the foot about its longitudinal axis is obtained. Such foot positions are desired for example in the manufacture of orthopaedic footwear which is intended to correct an oblique position of the pelvis.

FIG. 1 also schematically shows an evaluation unit 30, the evaluation unit containing a database in which desired ranges for the measured values of the pressure sensors are stored depending on different desired positions. The evaluation unit can be a calculator, for example.

The arrangement further comprises a graphic output device preferably coupled to the evaluation unit, on which depending on the values measured by the sensors, the data relating to the spatial shape of the foot/feet and the anatomic and/or medical data of the customer, proposals are made for a best-fitting shoe and/or for an individually manufactured shoe.

The values measured by the sensors can be transmitted to the evaluation unit directly via a connection or can be input by the user using a human-machine-interface.

According to the invention, a desired position is first determined with the customer, which is predefined by the footwear to be selected or manufactured. In the embodiment, a number of proposals are thus made to the customer using a first database, which contain information on the available or fabricable shoe shapes and contain desirable pressure distributions depending on the individual anatomy of the customer. According to the invention, the access to this database is performed using all or a selection from the following criteria:

gender of the customer

a pre-selection of the desired shoe shape by the customer

possible restrictions of use specified by the customer such as

running shoe, dancing shoe, outdoor shoe, sports shoe etc.

anatomic measured values such as total body weight, general geometry of the foot,

the latter being entered by means of an input medium such as a keyboard or a tactile field or being automatically determined from a 3D digitalization of the foot itself in a basic position. When the desired position or desired positions is/are determined in this way, the foot of the customer is then moved to the appropriate position(s) determined from the database, and the digitalization of the foot necessary for the selection of a best-fitting shoe or the manufacture of an individual shoe is preformed in this optimum position with respect to the shoe style and the anatomy.

The desired position can be predefined by geometric specifications or by values measured by the sensors. Preferably, both specifications are taken into account. Usually, it will often be impossible to obtain anatomically desired pressure ratios for all possible fashionable shoe styles. Therefore, if the predefined values measured by the sensors are not reached for a specific geometric position, a different footwear which is better adapted to the individual anatomical facts is then proposed to the customer.

In an embodiment, the foot is moved to different spatial positions during digitizing, and a respective detail of the spatial form is determined for the respective foot position. It is thus possible, for example, to sense the foot in different spatial positions in a left and right lateral image, and therefore to determine, for example, the change in the foot length as a function of the spatial position from one single digitizing operation, which means a gain in time.

FIG. 2 shows the arrangement of FIG. 1 when adapted to a smaller female foot 16′. For this purpose, the supports 12 and 14 were shifted parallel to the bottom plate 10, as indicated by the arrows 32 and 34. Preferably, this shifting is performed using a motor.

The invention was described with reference to a preferred embodiment. This use of the digitalization of a female foot is to be understood as an example only and does not constitute a restriction of the inventive idea. The use of more than two vertical supports, a different mechanical configuration of the device for changing the position of the foot, applications in the fields of orthopaedic shoes and boots, sports footwear and medical diagnostics and research in the field of foot medicine are covered by the inventive idea.

Compared with the prior art relating to the optical 3D digitalization of feet, it is essential for the inventive idea that the foot is moved to a desired spatial position before it is digitized, the spatial position resulting from the desired geometric position and/or from physical measured values, in particular from pressure values.

A further inventive idea, in particular for the individual manufacture or the best-fit selection of orthopaedic footwear, consists in selecting from a set of spatial shapes determined in different desired positions, those spatial shapes which constitute an optimum compromise between the anatomically/therapeutically desirable shape, the availability and the manufacturing costs.

1. A method for the three-dimensional, digitized sensing of the spatial shape of at least one foot or of both feet of a person for a selection or individual manufacture of footwear, the method comprising the following steps:
determining a desired position for the foot/feet which is predefined by the footwear to be selected or manufactured;
moving the foot/feet to the desired position by means of a device which can be adjusted; and
optical sensing and digitizing the three-dimensional spatial shape of the foot/feet in the desired position.

2. The method according to claim 1, wherein the foot/feet is/are successively moved to different desired positions and the three-dimensional spatial shape is determined in each of these positions.

3. The method according to claim 1, wherein the desired position is predefined by geometric specifications.

4. The method according to claim 1, wherein at least one physical sensor is mounted in the device and a value measured by the sensor is predefined for the desired position.

5. The method according to claim 4, wherein a plurality of sensors is provided and the sensors provide static and/or dynamic pressure values and/or temperature values and/or electrodynamic values at different points of the sole of the foot.

6. The method according to claim 4, wherein the sensors provide measured values relating to the posture of the person.

7. The method according to claim 1, wherein the foot/feet is/are moved to a desired position in which the heel is higher than the front toe plateau.

8. The method according to claim 1, wherein the foot is tilted along its longitudinal axis to obtain a desired position.

9. The method according to claim 1, wherein the desired position/the desired positions is/are taken from a first database depending on at least one of the following specifications:
gender of the person;
pre-selection of the desired shoe shape;
possible restrictions of use; and
anatomic measured values.

10. The method according to claim 1, wherein a selection of the footwear is performed with the aid of a second database in which rules for the selection or manufacture of a best-fitting shoe are stored depending on the determined spatial shape and/or data derived therefrom, the customer demands, the anatomy of the customer, medical rules and technical shoe manufacturing facts.

11. An arrangement by means of which a foot or both feet of a person is/are moved to a desired position in which the spatial shape of the foot/feet is digitized in a mainly contactless manner, the arrangement comprising:
a bottom plate having a support surface on which the front part of the foot rests;
a first support on which the arch of the foot rests; and
a second support on which the heel rests;
the first and second supports being movable parallel and perpendicular to the bottom plate to move the foot/feet to the desired position.

12. The arrangement according to claim 11, wherein both supports each have two separately movable parts which are arranged relative to each other such that the foot is tilted about its longitudinal axis in case of a different height of the respective two parts.

13. The arrangement according to claim 11, wherein the first and second supports are realized by pneumatically and/or hydraulically deformable hollow bodies.

14. The arrangement according to claim 11, wherein a first pressure sensor is provided in the support surface of the bottom plate, at least one second pressure sensor is provided in the first support and at least one third pressure sensor is provided in the second support, the pressure sensors being suitable for measuring the resting pressure of the front part of the foot, the arch of the foot and the heel, respectively.

15. The arrangement according to claim 14, further comprising an evaluation unit, the evaluation unit containing a database in which desired ranges for the values measured by the pressure sensors are stored depending on the desired position.

16. The arrangement according to claim 13, further comprising a graphic output device on which proposals for a best-fitting shoe and/or for an individually manufactured shoe are made depending on the values measured by the sensors, the data relating to the spatial shape of the foot/feet and the anatomic and/or medical data of the customer.

17. The arrangement according to claim 11, wherein at least the major part of the bottom plate, of the first support and of the second support is made of a transparent material.
